(19) United States
(12) Patent Application Publication
(10) Pub. No.: US 2026/0069165 A1
(43) Pub. Date: Mar. 12, 2026

(54) METHOD FOR DETERMINING FOOT DIMENSION DATA

(71) Applicant: DELTA SHOE TECH LTD., Taichung City (TW)

(72) Inventor: Tsung-E LIAO, Taichung City (TW)

(21) Appl. No.: 19/006,693

(22) Filed: Dec. 31, 2024

(30) Foreign Application Priority Data
Sep. 12, 2024 (TW) 113134709

(51) Int. Cl. A61B 5/107; G06T 7/60; G06T 7/73

(52) U.S. Cl. CPC A61B 5/1074; A61B 5/1075; G06T 7/60; G06T 7/73; G06T 2207/30196

(57) ABSTRACT

A method for determining foot dimension data includes: receiving a top-view foot image and a hindfoot image; obtaining first position data and second position data; obtaining a first foot contour in the top-view foot image; obtaining a second foot contour in the hindfoot image; determining an anterior reference point on the first foot contour; determining a posterior reference point on the second foot contour; adjusting the second foot contour to obtain an adjusted foot contour that has an adjusted posterior reference point; superimposing the adjusted foot contour on the first foot contour; obtaining a first straight line; obtaining a lateral point and a medial point; and determining the foot dimension data of the foot at least based on the lateral point, the medial point and the adjusted posterior reference point.

### CROSS-REFERENCE TO RELATED APPLICATION

This application claims priority to Taiwanese Invention Patent Application No. 113134709, filed on Sep. 12, 2024, the entire disclosure of which is incorporated by reference herein.

### FIELD

The disclosure relates to a method for determining foot dimension data of a foot of a subject.

### BACKGROUND

A shoe last is shaped like a human foot, and is used by shoemakers and cordwainers in the manufacturing and repair of shoes. Shoe lasts come in many styles and sizes. A preset size of a shoe last usually corresponds to a preset length range and a preset width range, is the basis of a shoe size of a shoe, and has influence on shoe volume (which refers to the overall space that a human foot can take up inside the shoe). A shoe fits a person properly when a sole length and a sole width of his/her foot fall respectively in the preset length range and the preset width range to which the shoe last for making the shoe corresponds.

Conventionally, a sole length and a sole width for a person is manually measured by a shoemaker. However, such approach is prone to errors by virtue of manual measurement.

Currently, a three-dimensional (3D) scanner can be utilized to build a 3D model of a foot of a person, and a sole length and a sole width for the person can be determined according to the 3D model thus built. However, such approach consumes relatively more computational resources and requires a computing device (e.g., a processor, a computer or a server) that has a relatively high computing power.

### SUMMARY

Therefore, an object of the disclosure is to provide a method for determining foot dimension data of a foot of a subject that can alleviate at least one of the drawbacks of the prior art.

According to the disclosure, the method is adapted to be implemented by a computing device, and includes steps of:
receiving a top-view foot image and a hindfoot image, the top-view foot image having an anterior part of the foot that at least includes a plurality of toes of the foot and a plurality of heads respectively of a plurality of metatarsal bones of the foot, the hindfoot image having a posterior part of the foot that at least includes a heel of the foot, at least one part of the foot contained in the top-view foot image being also contained in the hindfoot image;
obtaining plural pieces of first position data, the pieces of first position data being related respectively to a plurality of reference points and respectively representing plural locations in the top-view foot image;
obtaining plural pieces of second position data, the pieces of second position data being related respectively to the reference points and respectively representing plural locations in the hindfoot image;
identifying a contour of the foot contained in the top-view foot image to obtain a first foot contour;
identifying a contour of the foot contained in the hindfoot image to obtain a second foot contour;
determining an anterior reference point on the first foot contour;
determining a posterior reference point on the second foot contour;
based on a relationship between the pieces of first position data and the pieces of second position data, adjusting the second foot contour to obtain an adjusted foot contour that has an adjusted posterior reference point corresponding to the posterior reference point;
superimposing the adjusted foot contour on the first foot contour;
obtaining a first straight line that passes through the anterior reference point and the adjusted posterior reference point;
obtaining, based on the first straight line, a lateral point and a medial point on the first foot contour, the lateral point and the medial point being two farthest points from the first straight line respectively at two sides of the first straight line; and
determining the foot dimension data of the foot at least based on the lateral point, the medial point and the adjusted posterior reference point.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a schematic image illustrating an example of a top-view foot image according to an embodiment of the disclosure.

FIG. 2 is a schematic image illustrating an example of a hindfoot image according to an embodiment of the disclosure.

FIG. 3 is a schematic image illustrating an example of a first foot contour according to an embodiment of the disclosure.

FIG. 4 is a schematic image illustrating an example of a second foot contour according to an embodiment of the disclosure.

FIG. 5 is a schematic image illustrating an example of superimposing an adjusted foot contour on the first foot contour according to an embodiment of the disclosure.

FIG. 6 is a schematic image illustrating an example of a sole width and a post-toes segment length according to an embodiment of the disclosure.

FIG. 7 is a schematic image illustrating an example of a sole length according to an embodiment of the disclosure.

FIG. 8 is a flow chart illustrating a method for determining foot dimension data of a foot of a subject according to an embodiment of the disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

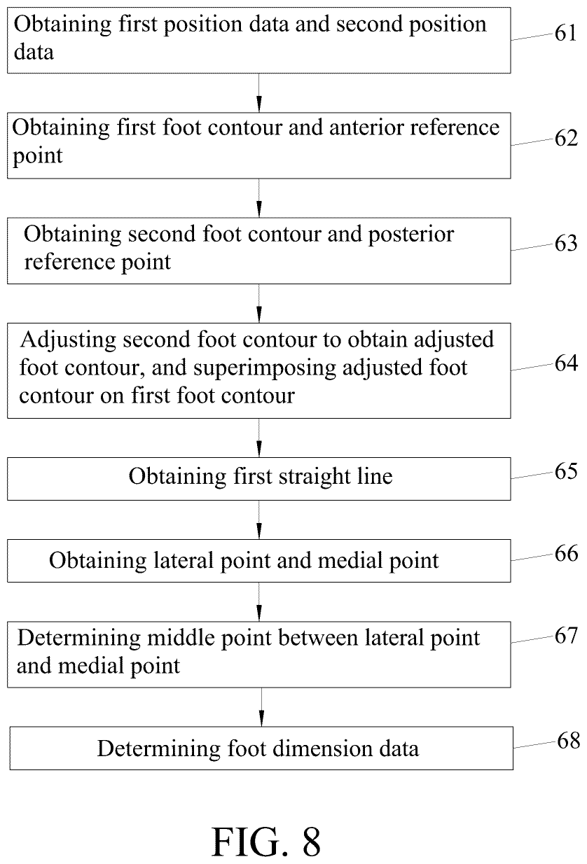

Referring to FIG. 8, an embodiment of a method for determining foot dimension data of a foot (which may be a left foot or a right foot) of a subject (e.g., a human) according to the disclosure is illustrated. The method is adapted to be implemented by a computing device. For example, the method is implemented as a software program for being executed by the computing device. The computing device may be implemented to be a smartphone, a computing server, a desktop computer, a laptop computer, a notebook computer or a tablet computer, but implementation thereof is not limited to what are disclosed herein and may vary in other embodiments. The method includes steps 61 to 68 delineated below.

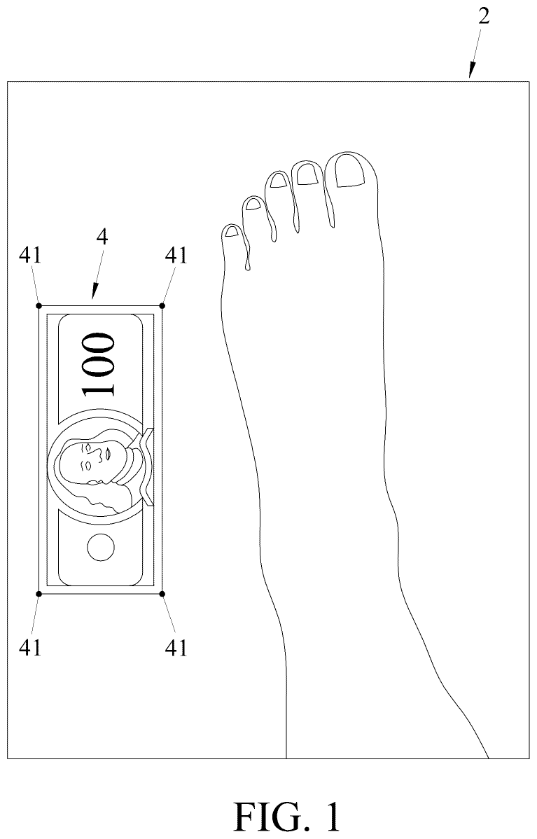

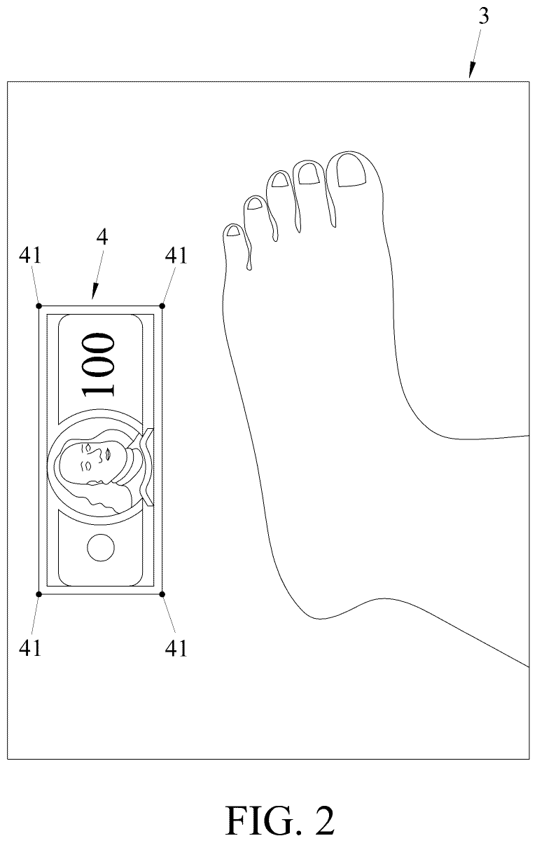

In step 61, the computing device receives a top-view foot image 2 (see FIG. 1) and a hindfoot image 3 (see FIG. 2). The top-view foot image 2 has an anterior part of the foot that at least includes a plurality of toes of the foot and a plurality of heads respectively of a plurality of metatarsal bones of the foot. The hindfoot image 3 has a posterior part of the foot that at least includes a heel of the foot. At least one part of the foot contained in the top-view foot image 2 is also contained in the hindfoot image 3.

As shown in FIG. 1, an example of the top-view foot image 2 is illustrated. The top-view foot image 2 is captured from the top of the anterior part of the foot, and the entirety of all toes of the foot are contained in the top-view foot image 2. In particular, at least a region defined by five tips respectively of five toes and five heads respectively of five metatarsal bones of the foot is contained in the top-view foot image 2. In some embodiments, only the anterior half of the foot is contained in the top-view foot image 2. In some embodiments, the entirety of the foot is contained in the top-view foot image 2 as shown in FIG. 1.

As shown in FIG. 2, an example of the hindfoot image 3 is illustrated. The hindfoot image 3 is captured from an upper-lateral side of the posterior part of the foot, and the entirety of a heel of the foot is contained in the hindfoot image 3. In particular, a calcaneal region at least including the back of the heel is contained in the hindfoot image 3. In some embodiments, only the posterior half of the foot is contained in the hindfoot image 3. In some embodiments, the entirety of the foot is contained in the hindfoot image 3 as shown in FIG. 2.

Then, the computing device obtains plural pieces of first position data, wherein the pieces of first position data are related respectively to a plurality of reference points of a reference object and respectively represent plural locations in the top-view foot image. Similarly, the computing device obtains plural pieces of second position data, wherein the pieces of second position data are related respectively to the reference points of the reference object and respectively represent plural locations in the hindfoot image. In this embodiment, the reference object is a rectangular banknote 4, and four corners 41 of the rectangular banknote 4 serve respectively as the reference points. The rectangular banknote 4 may be a banknote of any country (e.g., United States, Japan or the like). It should be noted that the reference object is not limited to the disclosure herein and may vary in other embodiments. In some embodiments, the reference object is a physical object (e.g., the rectangular banknote 4) having predefined dimensions and a predefined shape. In some embodiments, the reference object is a virtual object having predefined dimensions and a predefined shape. That is to say, the reference object may be any item that has a predefined size and a predefined shape. In this embodiment, the computing device utilizes techniques of image recognition to identify the reference object in each of the top-view foot image 2 and the hindfoot image 3. Then, the computing device determines pixel coordinates of each of the reference points in the top-view foot image 2 as the pieces of first position data, and determines pixel coordinates of each of the reference points in the hindfoot image 3 as the pieces of second position data.

It is worth noting that in an embodiment where each of the top-view foot image 2 and the hindfoot image 3 does not contain the reference object, an image capturing device (e.g., a digital camera) that captures the top-view foot image 2 and the hindfoot image 3 would determine plural reference points based on the top-view foot image 2 as the pieces of first position data, determine another plural reference points based on the hindfoot image 3 as the pieces of second position data, and send the pieces of first position data and the pieces of second position data to the computing device.

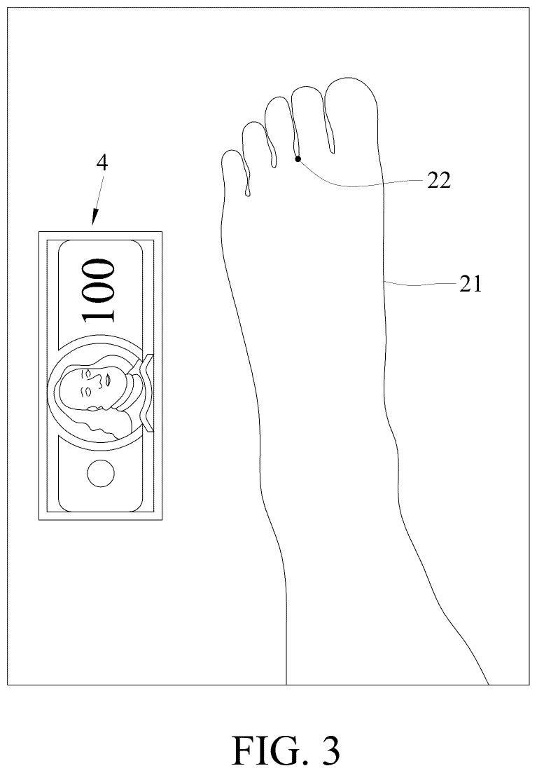

Referring to FIG. 3, in step 62, the computing device identifies a contour of the foot contained in the top-view foot image 2 to obtain a first foot contour 21, and determines an anterior reference point 22 on the first foot contour 21. Specifically, the computing device determines a portion of the first foot contour 21 that is related to interdigital spaces of the foot, and selects, within the portion of the first foot contour 21 thus determined, a trough point on the first foot contour 21 that is related to a posterior point of a second interdigital space of the foot as the anterior reference point 22.

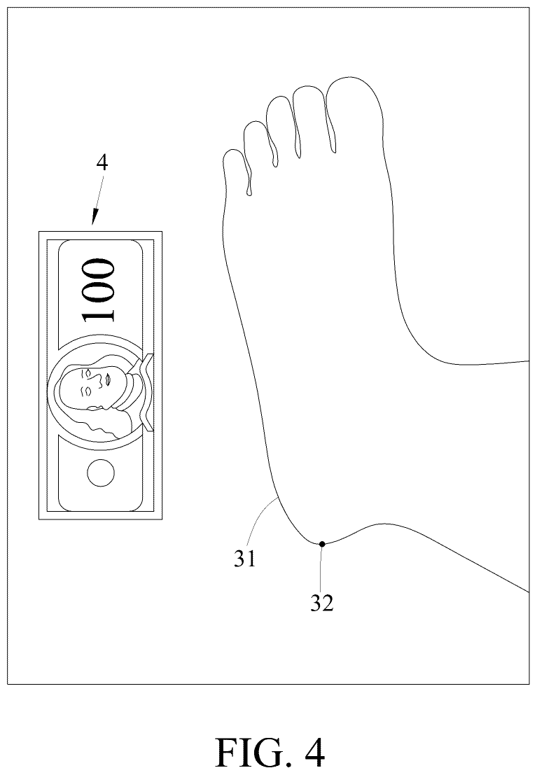

Referring to FIG. 4, in step 63, the computing device identifies a contour of the foot contained in the hindfoot image 3 to obtain a second foot contour 31, and determines a posterior reference point 32 on the second foot contour 31.

It is worth noting that under a framework of “Grounded-Segment-Anything” which is published by the International Digital Economy Academy (IDEA) on a developer platform of “GitHub” (please refer to https://github.com/idea-research), and which combines an object detection model of “Grounding DINO” and an image segmentation model of “Segment Anything”, the computing device performs image segmentation on each of the top-view foot image 2 and the hindfoot image 3, to obtain a mask of the foot for said each of the top-view foot image 2 and the hindfoot image 3. Then, the computing device calls a function of “findContours” provided by a library of “OpenCV” to obtain the first foot contour 21 based on the mask obtained for the top-view foot image 2, and to obtain the second foot contour 31 based on the mask obtained for the hindfoot image 3. In addition, the computing device calls functions of “adaptiveThreshold” and “morphologyEx” provided by the library of “OpenCV” to obtain a mask of the portion of the first foot contour 21 that is related to the interdigital spaces, calls the function of “findContours” to obtain a contour of the interdigital spaces, and then selects the anterior reference point 22 on the contour of the interdigital spaces. Moreover, according to plane geometry, the computing device calls at least functions of “fitLine” and “minAreaRect” provided by the library of “OpenCV” to facilitate in determining a most prominent point on the second foot contour 31 as the posterior reference point 32.

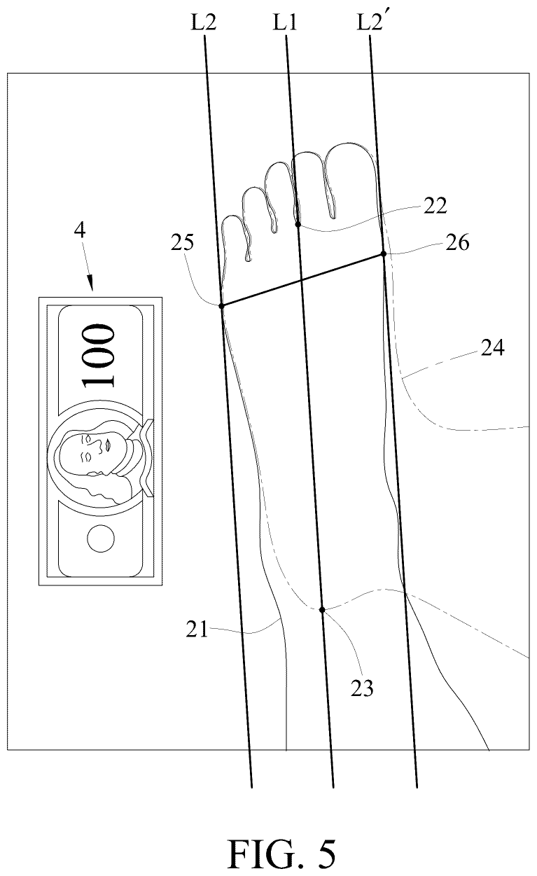

In step 64, the computing device adjusts the pieces of second position data to conform respectively with the pieces of first position data so as to obtain a transformation function expressing a relationship between the pieces of first position data and the pieces of second position data. Then, the computing device adjusts the second foot contour 31 by applying the transformation function thereto to obtain an adjusted foot contour 24 (see FIG. 5) that has an adjusted posterior reference point 23 corresponding to the posterior reference point 32. In particular, the computing device calls a function of “estimateAffinePartial2D” provided by the library of “OpenCV” to adjust the pieces of second position data (which are related to the four corners of the rectangular banknote in the hindfoot image) to conform respectively with the pieces of first position data (which are related to the four corners of the rectangular banknote in the top-view foot image) so as to obtain the transformation function, and to apply the transformation function to the second foot contour 31 to obtain the adjusted foot contour 24. In this way, the second foot contour 31 would be adjusted to form the adjusted foot contour 24.

After that, as shown in FIG. 5, the computing device superimposes the adjusted foot contour 24 on the first foot contour 21.

In step 65, the computing device obtains a first straight line (L1) that passes through the anterior reference point 22 and the adjusted posterior reference point 23. That is to say, the first straight line (L1) can be determined based on pixel coordinates of the anterior reference point 22 and pixel coordinates of the adjusted posterior reference point 23.

Referring to FIG. 5, in step 66, the computing device obtains, based on the first straight line (L1), a lateral point 25 and a medial point 26 on the first foot contour 21 that are two farthest points from the first straight line (L1) respectively at two sides of the first straight line (L1). In this embodiment, the lateral point 25 is a tangent point where a first tangent line (L2) that is parallel to the first straight line (L1) and that is spaced apart from the first straight line (L1) in a lateral direction just touches the first foot contour 21, and the medial point 26 is a tangent point where a second tangent line (L2′) that is parallel to the first straight line (L1) and that is spaced apart from the first straight line (L1) in a medial direction just touches the first foot contour 21. The lateral point 25 and the medial point 26 thus obtained are regarded respectively as two farthest points from the first straight line (L1) respectively at two sides of the first straight line (L1).

It is worthy of note that since a foot is relatively wider in an anterior portion thereof, in some embodiments, the computing device selects the lateral point 25 and the medial point 26 within an anterior half part of the first foot contour 21 where a part of the first foot contour 21 that corresponds to the heel of the foot is omitted.

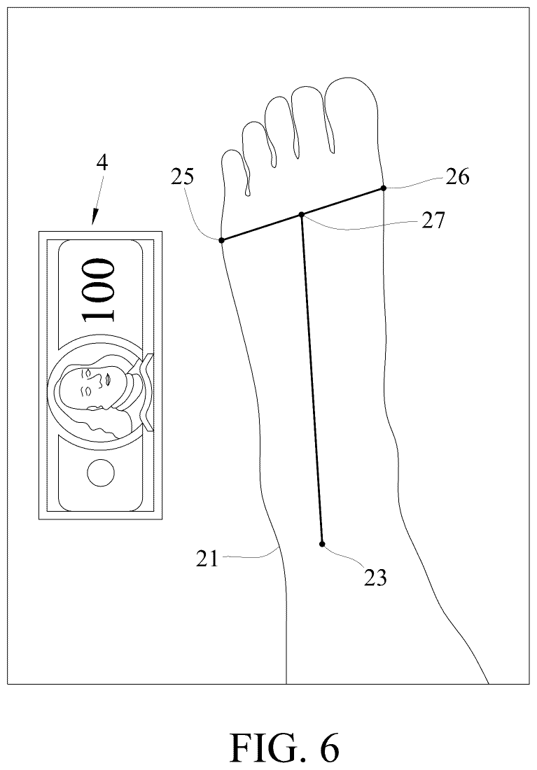

Referring to FIG. 6, in step 67, the computing device determines a middle point 27 between the lateral point 25 and the medial point 26.

In step 68, the computing device determines the foot dimension data of the foot at least based on the lateral point 25, the medial point 26 and the adjusted posterior reference point 23.

Referring to FIG. 6, in one embodiment where determining the foot dimension data of the foot is to determine a post-toes segment length, the computing device computes a distance between the middle point 27 and the adjusted posterior reference point 23 as the post-toes segment length.

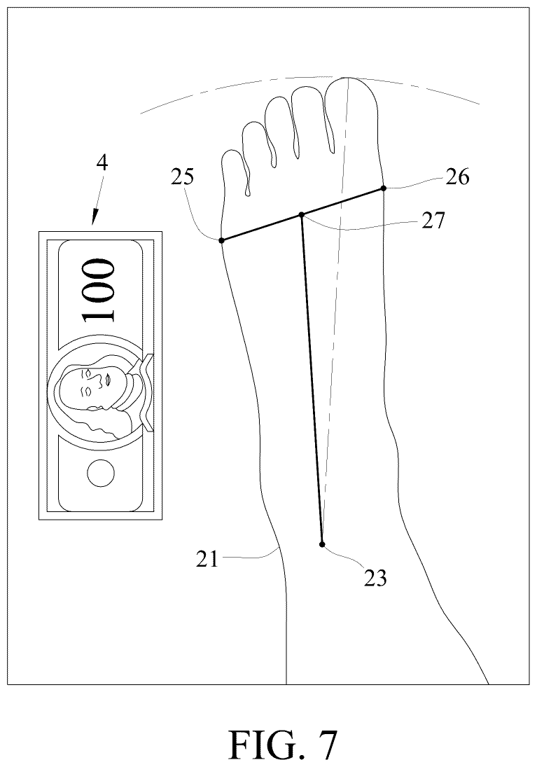

Referring to FIG. 7, in one embodiment where determining the foot dimension data of the foot is to determine a sole length, the computing device selects, on the first foot contour 21, a distal point that is farthest from the adjusted posterior reference point 23, and computes a distance between the distal point and the adjusted posterior reference point 23 as the sole length. More specifically, the computing device obtains a circumcircle of the first foot contour 21 with the adjusted posterior reference point 23 as a center, and selects a tangent point where the circumcircle just touches the first foot contour 21 as the distal point.

In one embodiment where determining the foot dimension data of the foot is to determine a sole width, the computing device computes a distance between the lateral point 25 and the medial point 26 as the sole width.

It is worth noting that the foot dimension data such as the sole length, the sole width and the post-toes segment length may serve as references for making shoes. Alternatively, the foot dimension data may serve as the basis of suggestions provided by a clerk of a shoe shop to customers. For example, the clerk may help a customer select shoes that properly fit the customer (i.e., shoes with the right size and shape for the customer's feet). At the same time, a customer may utilize the foot dimension data to choose shoes that fit properly for himself/herself on an online shop or in a physical store.

To sum up, for the method for determining foot dimension data of a foot of a subject according to the disclosure, the computing device is utilized to adjust a hindfoot image of the foot and then to superimpose the hindfoot image thus adjusted on a top-view foot image of the foot to obtain a superimposed image. Thereafter, the computing device is further utilized to determine the lateral point 25, the medial point 26 and the adjusted posterior reference point 23 based on the superimposed image. Subsequently, the computing device is further utilized to obtain the foot dimension data (e.g., the sole length, the sole width or the post-toes segment length) of the foot at least based on the lateral point 25, the medial point 26 and the adjusted posterior reference point 23. Compared to a conventional approach where a 3D scanner is involved in determining foot dimension data, the method according to the disclosure has a relatively low computational complexity. Thus, the computing device is only required to have a relatively average computing power, and thereby hardware costs may be lowered. It is worthy of note that compared to an approach where foot dimension data are determined based on the adjusted posterior reference point 23, and a lateral extremely-prominent point and a medial extremely-prominent point on the first foot contour 21, determining the foot dimension data based on the lateral point 25, the medial point 26 and the adjusted posterior reference point 23 may be better in view of repeatability and reproducibility of measurements (i.e., multiple foot dimension data derived from multiple times of measurements performed on a single person would have a relatively low numerical variation).

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for determining foot dimension data of a foot of a subject, adapted to be implemented by a computing device, the method comprising:
receiving a top-view foot image and a hindfoot image, the top-view foot image having an anterior part of the foot that at least includes a plurality of toes of the foot and a plurality of heads respectively of a plurality of metatarsal bones of the foot, the hindfoot image having a posterior part of the foot that at least includes a heel of the foot, at least one part of the foot contained in the top-view foot image being also contained in the hindfoot image;
obtaining plural pieces of first position data, the pieces of first position data being related respectively to a plurality of reference points of a reference object and respectively representing plural locations in the top-view foot image;
obtaining plural pieces of second position data, the pieces of second position data being related respectively to the plurality of reference points of the reference object and respectively representing plural locations in the hindfoot image;
identifying a contour of the foot contained in the top-view foot image to obtain a first foot contour;
identifying a contour of the foot contained in the hindfoot image to obtain a second foot contour;
determining an anterior reference point on the first foot contour;
determining a posterior reference point on the second foot contour;
based on a relationship between the pieces of first position data and the pieces of second position data, adjusting the second foot contour to obtain an adjusted foot contour that has an adjusted posterior reference point corresponding to the posterior reference point;
superimposing the adjusted foot contour on the first foot contour;
obtaining a first straight line that passes through the anterior reference point and the adjusted posterior reference point;
obtaining, based on the first straight line, a lateral point and a medial point on the first foot contour, the lateral point and the medial point being two farthest points from the first straight line respectively at two sides of the first straight line; and
determining the foot dimension data of the foot at least based on the lateral point, the medial point and the adjusted posterior reference point.

2. The method as claimed in claim 1, wherein determining an anterior reference point includes:
determining a portion of the first foot contour that is related to interdigital spaces of the foot; and
selecting, within the portion of the first foot contour thus determined, a trough point on the first foot contour that is related to a posterior point of a second interdigital space of the foot as the anterior reference point.

3. The method as claimed in claim 1, wherein determining the foot dimension data of the foot is to determine a post-toes segment length, and includes determining a middle point between the lateral point and the medial point, and computing a distance between the middle point and the adjusted posterior reference point as the post-toes segment length.

4. The method as claimed in claim 1, wherein determining the foot dimension data of the foot is to determine a sole length, and includes selecting, on the first foot contour, a distal point that is farthest from the adjusted posterior reference point, and computing a distance between the distal point and the adjusted posterior reference point as the sole length.

5. The method as claimed in claim 4, wherein selecting the distal point includes obtaining a circumcircle of the first foot contour with the adjusted posterior reference point as a center, and selecting a tangent point where the circumcircle just touches the first foot contour as the distal point.

6. The method as claimed in claim 1, wherein determining the foot dimension data of the foot is to determine a sole width, and includes computing a distance between the lateral point and the medial point as the sole width.

7. The method as claimed in claim 1, wherein the reference object is a physical object having predefined dimensions and a predefined shape.

8. The method as claimed in claim 7, wherein the reference object is a rectangular banknote, and four corners of the rectangular banknote serve respectively as the reference points.

9. The method as claimed in claim 1, wherein determining plural pieces of first position data is to determine pixel coordinates of each of the reference points in the top-view foot image.

10. The method as claimed in claim 1, wherein determining plural pieces of second position data is to determine pixel coordinates of each of the reference points in the hindfoot image.

11. The method as claimed in claim 1, wherein the lateral point is a tangent point where a first tangent line that is parallel to the first straight line and that is spaced apart from the first straight line in a lateral direction just touches the first foot contour, and the medial point is a tangent point where a second tangent line that is parallel to the first straight line and that is spaced apart from the first straight line in a medial direction just touches the first foot contour.

12. The method as claimed in claim 1, wherein adjusting the second foot contour includes adjusting the pieces of second position data to conform respectively with the pieces of first position data so as to obtain a transformation function expressing the relationship between the pieces of first position data and the pieces of second position data, and applying the transformation function to the second foot contour to obtain the adjusted foot contour.
